(19) United States
(12) Patent Application Publication
(10) Pub. No.: US 2023/0386660 A1
(43) Pub. Date: Nov. 30, 2023

(54) SYSTEM AND METHOD FOR DETECTING GASTROINTESTINAL DISORDERS

(71) Applicant: JUBAAN LTD, Yokne'am lllit (IL)

(72) Inventors: Asaf GOLAN, Zichron Yaakov (IL); David RAINIS, Haifa (IL); Ariel SCHIFF, Haifa (IL)

(21) Appl. No.: 18/029,151

(22) PCT Filed: Oct. 4, 2021
(86) PCT No.: PCT/IL2021/051189

§ 371 Date: Mar. 29, 2023

Related U.S. Application Data
(60) Provisional application No. 63/087,401, filed on Oct. 5, 2020.

(51) Int. Cl. G16H 50/20; G16H 30/40; G06T 7/00

(52) U.S. Cl. CPC G16H 50/20; G16H 30/40; G06T 7/0012; G06T 2207/20081

(57) ABSTRACT

A system comprising at least one hardware processor and a non-transitory computer-readable storage medium having stored thereon program code, the program code executable by the at least one hardware processor to receive n images, each depicting a tongue of a subject, preprocess then images, wherein the preprocessing comprises at least one of image selection and image adjustment, thereby obtaining n′ images, produce m presentations of each of the n′ images using at least one feature enhancing algorithm, classify the n′*m presentations into classes by applying a machine learning algorithm on the n′*m presentations, wherein the classes comprise at least a positive for gastrointestinal disorders and a negative for gastrointestinal disorders, and identify the subject as suffering from a gastrointestinal disorder when at least a predetermined fraction/percentage of the n′*m presentations are classified as being positive for gastrointestinal disorders.

### FIELD OF THE INVENTION

The present invention, in some embodiments thereof, relates to tongue diagnosis and, more particularly, but not exclusively, to detection of gastrointestinal disorders.

### BACKGROUND

Tongue diagnosis is a common diagnostic tool in traditional Chinese medicine. Observation of a tongue of a subject enables practitioners to diagnose symptoms and/or pathologies of the subject. Some of the characteristics of the tongue which are observed by the practitioners are shape, color, texture, geometry, and morphology. By observing such characteristics, practitioners are able to detect pathologies of the subject in a non-invasive manner.

Today, the commonly used methods for detection of lower gastrointestinal pathologies include the stool guaiac test; Fecal Occult Blood Test (FOBT), and the Fecal Immunochemical Test (FIT).

FIT uses specific antibodies to detect human blood in the stool it is more definitive for gastrointestinal pathologies than other types of stool tests such as the qualitative guaiac fecal occult blood test (FOBT). The Guaiac tests can often result in a false positive result due to other types of blood that may be in the digestive system as a result of diet (e.g. red meat) or certain medications. FIT is both more sensitive and specific than FOBT.

The FOBT or FIT generally have a sensitivity rate ranging between 40% and 70%. However, it is typically recommended that a subject is tested using the FOBT or FIT three times over the course of three consecutive days, in order to increase the sensitivity of the result. The cost of each of the kits usually ranges between $7 and $35 and the results of each lab analysis of the test takes about two weeks to receive.

Today, a common procedure for detecting upper gastrointestinal pathologies include gastroscopy, which involves insertion of a visual aid through an endoscope into the gastrointestinal tract. In order to identify bleeding in the upper gastrointestinal or other pathologies of the upper gastrointestinal, a subject therefore undergoes an invasive procedure. Preparation for such a procedure includes avoiding food and liquids for six to eight hours prior to the procedure.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

According to some embodiments of the present invention there is provided a system including at least one hardware processor, and a non-transitory computer-readable storage medium having stored thereon program code, the program code executable by the at least one hardware processor to: receive n images, each depicting a tongue of a subject, preprocess the n images, wherein the preprocessing includes at least one of image selection and image adjustment, thereby obtaining n′ images, produce m presentations of each of the n′ images using at least one feature enhancing algorithm, classify the n′*m presentations (or in other words, the m presentations of the n′ images) by applying a machine learning algorithm on the n′*m presentations, wherein the classes include at least a positive for gastrointestinal disorders and a negative for gastrointestinal disorders, and identify the subject as suffering from a gastrointestinal disorder when at least a predetermined fraction/percentage of the n′*m presentations are classified as being positive for gastrointestinal disorders.

According to some embodiments of the present invention there is provided a computer program product including a non-transitory computer-readable storage medium having program code embodied therewith, the program code executable by at least one hardware processor to: receive n images, each depicting a tongue or a portion of a tongue of a subject, preprocess the n images, wherein the preprocessing includes at least one of image selection and image adjustment, thereby obtaining n′ images, produce m presentations of each of the n′ images using at least one feature enhancing algorithm, classify the n′ images into at least two classes by applying a machine learning algorithm onto the n′*m presentations, wherein the at least two classes include positive for gastrointestinal disorders and negative for gastrointestinal disorders, and identify the subject as suffering from a gastrointestinal disorder when at least a predetermined fraction/percentage of the n′*m presentations are classified as being positive for gastrointestinal disorders.

According to some embodiments, the image selection includes movement detection wherein images captured during movement are assigned one or more motion vectors, followed by a sorting out of detected images in which the vector exceeds a predetermined threshold value. According to some embodiments, the image adjustment includes adjustment of one or more of contrast, brightness, level, hue, sharpness, and saturation of the n′ images.

According to some embodiments, the program code is executable to further subclassify the subject based, at least in part, on the n′*m presentations which are classified as being positive for gastrointestinal disorders, into one or more subclassifications of colon-related pathology and gastro-related pathology. According to some embodiments, the subclassification further includes two or more subclasses of colon-specific pathologies. According to some embodiments, two or more subclasses of colon-specific pathologies are selected from colorectal carcinoma (CRC), polyps, different types of polyps, and inflammatory bowel disease involving the lower intestinal tract (IBD).

According to some embodiments, the subclasses of the colon-specific pathologies are selected from adenomatous polyp, hyperplastic polyp, serrated polyp, inflammatory polyp, and villous adenoma polyp, and complex polyp. According to some embodiments, the subclassification further includes two or more subclasses of upper gastrointestinal specific pathologies. According to some embodiments, two or more subclasses of upper gastrointestinal-specific pathologies are selected from gastric malignancy, gastritis, esophageal malignancy, esophagitis and duodenitis.

According to some embodiments, the subclassification includes a score associated with a level of malignancy of a disorder. According to some embodiments, the subclassification includes a score corresponding with a potential chance of the subject developing malignancy in one or more pathologies. According to some embodiments, the m presentations can additionally include three dimensional presentations of the depicted tongue of the subject.

According to some embodiments, the program is configured to receive the n images from a plurality of different types of image capturing devices. According to some embodiments, the program is executable to normalize the received images. According to some embodiments, the hardware processor is couplable to at least one image capturing device and the program code is executable to identify a tongue of a subject in real time. According to some embodiments, the program code is executable to capture the n images.

There is provided, in accordance with some embodiments, a system including at least one hardware processor, and a non-transitory computer-readable storage medium having stored thereon program code, the program code executable by the at least one hardware processor to receive n images, each depicting a tongue (or a portion of the tongue) of a subject, preprocess the n images, wherein the preprocessing includes at least one of image selection and image adjustment, thereby obtaining n′ images, produce m presentations of each of the n′ images using at least one feature enhancing and/or extracting algorithm, classify the subject based, at least in part, on the n′*m presentations by applying a machine learning algorithm on the n′*m presentations and optionally additional data on the patient, wherein the classes include at least a positive for gastrointestinal disorders and a negative for gastrointestinal disorders, and identify the subject as suffering from a gastrointestinal disorders when at least a predetermined fraction/percentage of the n′*m presentations are classified as being positive for gastrointestinal disorders. According to some embodiments, the additional data on the patient may include, for example, one or more of blood pressure, heart rate, respiratory rate, age, gender, eating habits, ethnic background, and smoking habits of the subject.

In some embodiments, the image selection includes motion detection wherein images captured during movement are assigned one or more motion vectors, followed by a sorting out of detected images in which the vector exceeds a predetermined threshold value. In some embodiments, the image adjustment includes adjustment of one or more of contrast, brightness, texture, level, hue, and saturation of the n′ images and/or the n′*m presentations. In some embodiments, image adjustment includes creating additional image from part or all the n images, for example high dynamic range (HDR) imaging.

In some embodiments, the program code is executable to further subclassify the subject based, at least in part, on the n′*m presentations which are classified as being positive for gastrointestinal disorders into one or more subclassifications of colon-related pathology and gastro-related pathology. In some embodiments, the subclassification further includes two or more classes of colon-specific pathologies. In some embodiments, two or more subclasses of colon-specific pathologies are selected from colorectal carcinoma (CRC), polyps, and inflammatory bowel disease (IBD). In some embodiments, the subclassification further includes two or more classes of gastro-specific pathologies. In some embodiments, two or more subclasses of gastro-specific pathologies are selected from gastric malignancy, gastritis, esophageal malignancy, esophagitis and duodenitis.

In some embodiments, the m presentations include three dimensional presentations of the depicted tongue of the subject. In some embodiments, the program is configured to receive the n images from a plurality of different types of image capturing devices. In some embodiments, the program is executable to normalize the received images. In some embodiments, the m presentations include at least one of the original n images (or in other words, at least one of the captured n images). In some embodiments, the m presentations may be images. In some embodiments, the m presentations may include files in one or more image formats.

In some embodiments, the hardware processor is couplable to at least one image capturing device and the program code is executable to identify a tongue of a subject in real time. In some embodiments, the program code is executable to capture the n images.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is a schematic simplified illustration of a system for detection gastrointestinal disorders, in accordance with some embodiments of the present invention;

FIG. 2 is a flowchart of functional steps in a process for detection of gastrointestinal disorders, in accordance with some embodiments of the present invention;

FIG. 3 is a front view schematic illustration of an exemplary segmentation map, in accordance with some embodiments of the present invention; and

FIG. 4 is a perspective view simplified illustration of an exemplary image capturing device for detection of gastrointestinal disorders, in accordance with some embodiments of the present invention.

### DETAILED DESCRIPTION

According to an aspect of some embodiments of the present invention there is provided a system and method for detection of gastrointestinal disorders based on one or more images of a subject's tongue, using image processing, computer vision, color science and/or deep learning.

In some embodiments, the system comprises at least one hardware processor and a storage module having stored thereon a program code. In some embodiments, the program code is executable by the at least one hardware processor to receive n images, each depicting a tongue of a subject, and preprocess the n images, wherein the preprocessing comprises at least one of image selection and image adjustment, thereby obtaining n′ images. In some embodiments, the program code is executable to produce m presentations of each of the n′ images using at least one feature enhancing algorithm and classify the subject based, at least in part, on the n′*m presentations by applying a trained machine learning algorithm on the n′*m presentations. In some embodiments, the classes include at least a positive for gastrointestinal disorders and a negative for gastrointestinal disorders. In some embodiments, the program code is executable to identify the subject as suffering from a gastrointestinal disorders when at least a predetermined percentage of the n′*m presentations are classified as being positive for gastrointestinal disorders.

A potential advantage of the system and method disclosed herein is in that the detection of gastrointestinal disorders is based on one or more images of a tongue of a subject and is therefore non-invasive. In some embodiments, the detection of the gastrointestinal disorders is automatic. In some embodiments, the detection is analyzed in real time. In some embodiments, the system and method for the detection of gastrointestinal disorders detects the pathology at a sensitivity and/or specificity of at least 70%.

In some embodiments, the system and method for the detection of gastrointestinal disorders comprises using a generic or universal camera for obtaining an image of a tongue, based on which the pathology is then detected. In some embodiments, the system and method can be used by a user at home and/or without visiting a practitioner, for example, by using a smartphone camera to capture an image of their tongue. In some embodiments, the system and method for the detection of gastrointestinal disorders does not require any preparation or special diet during the day or days prior to obtaining an image of a tongue of a subject. With the exclusion that, according to some embodiments, consuming food products and/or beverages having pigments/dyes should be prevented prior to obtaining the image, such that the coloring of the consumed products does not mask the natural colors of the tongue of the subject.

A potential advantage to the system and method for the detection of gastrointestinal disorders not requiring any preparation or special diet during the day or days prior to obtaining an image of the tongue is in that a subject does not need to change eating habits in the day or days leading up to the examination. For example, oral medication prescribed to the subject can be taken regularly.

In some embodiments, the system and method for detection of gastrointestinal disorders is configured to replace commonly used detection tests such as, FOBT and FIT. In some embodiments, the system and method for detection of gastrointestinal disorders identifies at least three pathologies associated with the FOBT.

The present disclosure provides for one or more machine learning models trained to detect gastrointestinal disorders, which was developed by training deep neural networks using labeled images of tongues of subject with diagnosed gastrointestinal disorders. In some embodiments, the present machine learning models provide for greater prediction accuracy compared to known classification techniques. In some embodiments, the present disclosure employs deep learning techniques to generate automated, accurate and standardized machine learning models for early prediction of gastrointestinal disorders.

In some embodiments, the present disclosure provides for training one or more machine learning models, based, at least in part, on training data comprising image data depicting at least a portion of a tongue of a subject. In some embodiments, the image data comprises at least one of an image and a series of images. In some embodiments, the image data comprises at least one of a video segment, a motion vector segment, and a three-dimensional video segment. In some embodiments, the image data comprises the (original) captured images of the tongue (or portion of the tongue) of the subject. In some embodiments, the image data comprises one or more presentations of the one or more captured images of the tongue of the subject. In some embodiments, the presentations may be images. In some embodiments, the presentations may include files in one or more image formats.

In some embodiments, the image data depicts a tongue and/or a portion of a tongue of a subject. In some embodiments, and as described in greater detail elsewhere herein, the image data comprises data depicting at least one of the time at which the image data was captured and the relative time at which an image was captured in relation to the capture time of another image. In some embodiments, each possibility is a separate embodiment.

In some embodiments, the image data comprises n images per subject and/or tongue of a subject. In some embodiments, the n images comprise one or more images. In some embodiments, then images comprise a plurality of images. In some embodiments, the image data is obtained using an image sensor, such as an active-pixel sensor or a charge-coupled device. In some embodiments, the image data is obtained using an RGB imaging technique. In some embodiments, the image data is obtained using a digital topology technique. In some embodiments, the image data may be taken using different image capturing equipment. In some embodiments, the image data is obtained by focusing all or at least a portion of a reflected wavelength from the tongue of a subject. In some embodiments, the wavelengths range between 380 nm to 800 nm. In some embodiments, the reflected wavelength enables a depth of field of at least 100 mm.

According to some embodiments, and as described in greater detail elsewhere herein, the image data is obtained at a specified illumination of the tongue of the subject. In some embodiments, the image data is obtained while the tongue is illuminated at a specified illumination such that the tongue of the subject is obtained with an optimal color fidelity. In some embodiments, the tongue is illuminated using at least one of a laser and a filter configured to generate, at least in part, the specified illumination. In some embodiments, the system is configured to continuously calibrate the specified illumination.

According to some embodiments, the specified illumination is configured such that the colors of the tongue of the subject and the captured n images are metamerically matched. In some embodiments, and as described in greater detail elsewhere herein, the system is configured to change an illumination type, the illumination spectrum, and/or position of the illuminating element in order to maintain a metameric match between the colors of the tongue of the subject and the captured n images.

In some embodiments, the system for detection of gastrointestinal disorders is configured to receive, normalize, and/or compare different image data from various image capturing devices. In some embodiments, the image data is represented by n′ image data sets corresponding to the n images. In some embodiments, the n′ image data set is obtained by manipulating the n images in at least one of a pre-processing and an image processing analysis. In some embodiments, the image manipulation comprises at least one of color fidelity, texture enhancement, local contrast enhancement, local color contrast enhancement, geometric feature enhancement, image segmentation, image color segmentation, and motion detection. In some embodiments, each possibility is a separate embodiment.

In some embodiments, the image manipulation comprises at least one of a three dimensional representation of the tongue depicted by the image, a motion or movement detection associated with the movement of the tongue during the capturing of the image, and generation of a video using one or more of the n′ images and/or the n images. In some embodiments, each possibility is a separate embodiment. In some embodiments, the image data set comprises the manipulated n′ images. In some embodiments, the image manipulation comprises accounting for motion blur, distortion, and/or data replication caused by motion of the tongue during the capturing of the image.

In some embodiments, the system and method for detection of gastrointestinal disorders is configured to detect a gastrointestinal disorder based on at least a portion of the n′ images of the image data set using a trained machine learning module. In some embodiments, the machine learning module is trained on an image data set of n′ images associated with tongues of one or more subjects with one or more diagnosed gastrointestinal disorders.

In some embodiments, the machine learning module is configured to receive the n images of the image data and/or the n′ images of the image data set. In some embodiments, the machine learning module is trained to classify the received image data into classification and/or subclassifications associated with gastrointestinal disorders. In some embodiments, the system and/or method for detection of gastrointestinal disorders is configured to produce m presentations of each of the n′ images. In some embodiments, the system and/or method is configured to produce m presentations of at least a portion of the n′ images. In some embodiments, the machine learning module is configured to receive the m presentations. In some embodiments, the machine learning module is trained to classify the received m presentations into classification and/or subclassifications associated with gastrointestinal disorders.

In some embodiments, the classifications comprise at least one of negative for lower gastrointestinal pathologies, positive for lower gastrointestinal pathology, negative for upper gastrointestinal-related pathology, and positive for upper gastrointestinal-related pathology. In some embodiments, the subclassifications comprise at least one of colorectal carcinoma (CRC), polyps, inflammatory bowel disease (IBD), gastric malignancy, gastritis, esophageal malignancy, esophagitis and duodenitis. In some embodiments, each possibility is a separate embodiment.

According to some embodiments, the subclassification comprises types of pathologies, e.g., types of polyps. According to some embodiments, the subclassification comprises at least one of sessile polyps and pendunculated polyps. According to some embodiments, the subclassification comprises cancerous and/or non-cancerous polyps. According to some embodiments, the subclassification comprises benign and/or malignant polyp. According to some embodiments, the subclassification comprises one or more of adenomatous polyp, hyperplastic polyp, serrated polyp, inflammatory polyp, and villous adenoma polyp, and complex polyp.

According to some embodiments, the subclassification comprises ranking the identified disorder with a value associated with a risk level of the disorder. According to some embodiments, the risk level may be fatality level and/or urgency of a surgery. According to some embodiments, the subclassification comprises one or more ranges of sizes of polyps.

According to some embodiments, the subclassification comprises a score associated with a level of malignancy of a disorder. According to some embodiments, the subclassification comprises a score corresponding with a potential chance of the subject developing malignancy in one or more pathologies. For example, according to some embodiments, the subclassification may predict the chances of a subject having no polyps to develop one or more types of polyps. According to some embodiments, the score is evaluated in percentages, such as, for example, 0-10%, 10-30%, 30-50%, 50-70%, and over 70%. In some embodiments, the score is indicative of a chance of occurrence of a specific malignancy of a known disorder in a subject and/or a specific development of a malignancy of a disorder in a subject. According to some embodiments, the score is associated with a risk level of the subject developing malignancy in one or more pathologies. According to some embodiments, the score is associated with a risk level of the subject developing a pathology in the future.

In some embodiments, the system comprises at least one hardware processor and a non-transitory computer-readable storage medium having stored thereon program code. According to some embodiments, the program code is executable by the at least one hardware processor to receive n images, each depicting at least a portion of a tongue of a subject, preprocess then images, wherein the preprocessing comprises at least one of image selection and image adjustment, thereby obtaining n′ images, produce m presentations of each of the n′ images using at least one feature enhancing algorithm, classify the produced n′*m presentations into classes using a machine learning algorithm, wherein the classes comprise at least a positive for gastrointestinal disorders and a negative for gastrointestinal disorders, identify the subject as suffering from a gastrointestinal disorder when at least a predetermined fraction/percentage of the produced n′*m presentations are classified as being positive for gastrointestinal disorders.

According to some embodiments, and as described in greater detail elsewhere herein, the machine learning algorithm may be trained on a data set comprising n′*m presentations associated with one or more subjects. According to some embodiments, there is provided a computer program product comprising a non-transitory computer-readable storage medium having program code embodied therewith, the program code executable by at least one hardware processor to receive n images, each depicting a tongue of a subject, preprocess the n images, wherein the preprocessing comprises at least one of image selection and image adjustment, thereby obtaining n′ images, produce m presentations of each of the n′ images using at least one feature enhancing algorithm, classify the n′ images into at least two classes by applying a trained machine learning algorithm on the m presentations, wherein the at least two classes comprise positive for gastrointestinal disorders and negative for gastrointestinal disorders; and identify the subject as suffering from a gastrointestinal disorder when at least a predetermined fraction/percentage of the n′ images are classified as being positive for gastrointestinal disorders.

### System for Detection of Gastrointestinal Disorders

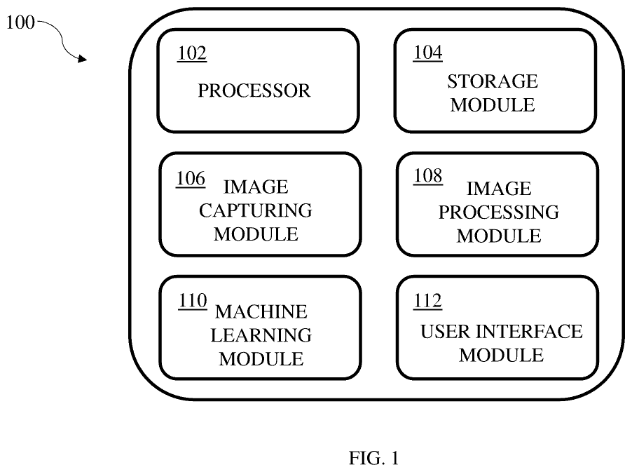

Reference is made to FIG. 1, which is a schematic simplified illustration of a system for detection of gastrointestinal disorders, in accordance with some embodiments of the present invention. In some embodiments, the system 100 comprises at least one of a hardware processor 102, a storage module 104, an image capturing module 106, an image processing module 108, a machine learning module 110, and a user interface module 112. In some embodiments, each possibility is a separate embodiment. In some embodiments, the system 100 is configured to detect gastrointestinal disorders of a subject based on an image of the tongue of the subject.

In some embodiments, the processor 102 is in communication with at least one of the storage module 104, the image capturing module 106, the image processing module 108, the machine learning model 110, and the user interface module 112. In some embodiments, the processor 102 is configured to control operations of any one or more of the storage module 104, the image capturing module 106, the image processing module 108, the machine learning model 110, and the user interface module 112. In some embodiments, each possibility is a separate embodiment.

In some embodiments, the storage module 104 comprises a non-transitory computer-readable storage medium. In some embodiments, the storage module 104 comprises one or more program code and/or sets of instructions for detection of gastrointestinal disorders wherein the program code instructs the use of at least one of the processor 102, the image capturing module 106, the image processing module 108, the machine learning module 110, and the user interface module 112. In some embodiments, each possibility is a separate embodiment. In some embodiments, the storage module 104 comprises one or more algorithms configured to detect gastrointestinal disorders of a subject, based on, at least in part, one or more images of a tongue of the subject using method 200.

In some embodiments, the image capturing module 106 is configured to obtain n images of a tongue of the subject. In some embodiments, the processor 102 commands the image capturing module 106 to obtain one or more of the n images. In some embodiments, the image capturing module 106 comprises an image capturing device and/or a coupler configured to communicate between system 100 and an image capturing device. For example, in some embodiments, the image capturing module 106 comprises a CMOS sensor. In some embodiments, the coupler comprises a cable or a wireless connection through which the processor 102 obtains the n images from an image capturing device.

In some embodiments, the image capturing module 106 is configured to illuminate the tongue of the subject during the capturing of the n images. In some embodiments, the processor 102 is configured to control the illumination of the image capturing module 106.

In some embodiments, the image capturing module 106 comprises and/or is in communication with one or more sensors configured to detect movement of a tongue of a subject. In some embodiments, the one or more sensors is a motion sensor, such as, for example, a thermal sensor.

In some embodiments, one or more of a program stored onto the storage module 104 is executable to capture the n images. In some embodiments, the processor 102 is configured to command the capture of the images in real time while receiving image data from the image capturing module 106. In some embodiments, the image capturing module 106 and/or system 100 are configured to receive images from a plurality of different types of image capturing devices. In some embodiments, and as described in greater detail elsewhere herein, the system 100 is configured to normalize different images which may be captured by more than one image capturing device using the image processing module 108.

In some embodiments, the processor 102 is in communication with a cloud storage unit. In some embodiments, the storage module 104 comprises a cloud storage unit. In some embodiments, the image processing module 108 is stored onto a cloud storage unit and/or the storage module 104. In some embodiments, the storage module 104 is configured to receive the n images by uploading the n images onto the couple storage unit of the storage module 104.

In some embodiments, the image processing module 108 is configured to preprocess the n images received using the image capturing module 106. In some embodiments, the image processing module 108 is configured to generate the image data set of n′ images based, at least in part, on the n images. In some embodiments, the image processing module is configured to apply image processing algorithms to at least apportion of the n images and/or the n′ images. In some embodiments, and as described in greater detail elsewhere herein, the image processing module 108 is configured to generate at least a portion of the n′ image data set using image processing algorithms.

In some embodiments, the machine learning module 110 receives the image data set of n′*m presentations. In some embodiments, and as described in greater detail elsewhere herein, the machine learning module 110 is trained to detect one or more gastrointestinal disorders associated with the image data set of n′*m presentations.

In some embodiments, the system 100 comprises a user interface module 112. In some embodiments, the user interface module 112 is configured to receive meta data from a user, such as, for example, age, gender, blood pressure, eating habits, risk factors associated with specific disorders, genetic data, medical history of the family of the subject and medical history of the subject. In some embodiments, the user interface module 112 communicates with the processor such that the user inputted data is fed to the machine learning module 110.

In some embodiments, the user interface module 112 comprises at least one of a display screen and a button. In some embodiments, the user interface module 112 comprises a software configured for transferring inputted information from a user to the processor 102. In some embodiments, the user interface module 112 comprises a computer program and/or a smartphone application.

In some embodiments, the user interface module 112 comprises a keyboard. In some embodiments, the user interface module is configured to receive data from the processor 102 and/or display data received from the processor 102. In some embodiments, the user interface module 112 is configured to display a result of a detection of a gastrointestinal disorder.

### Method for Detection of Gastrointestinal Disorders

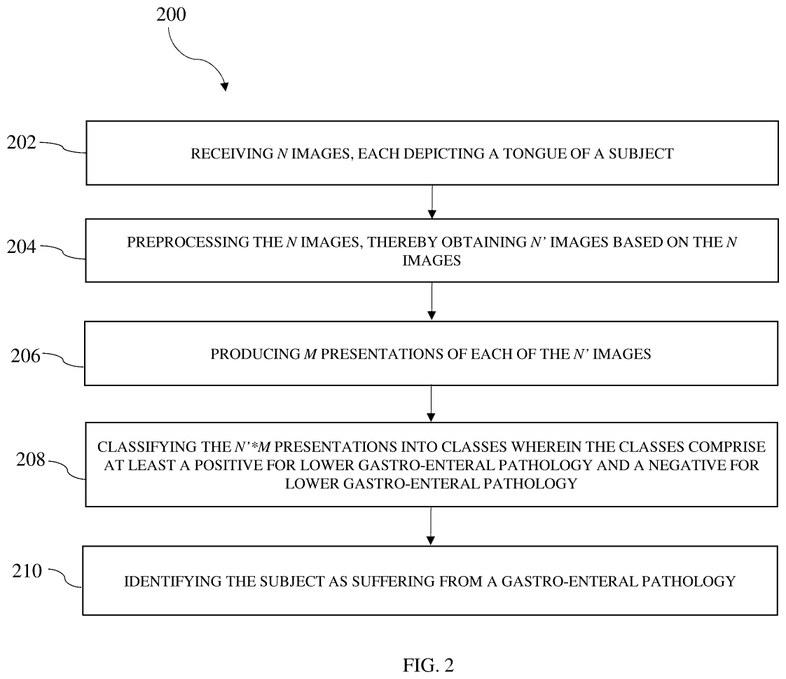

Reference is made to FIG. 2, which is a flowchart of functional steps in a process for detection of gastrointestinal disorders, in accordance with some embodiments of the present invention.

In some embodiments, the method 200 comprises obtaining one or more images of an oral cavity of a subject. In some embodiments, the method 200 comprises obtaining n images of a tongue of a subject. In some embodiments, the n images comprise at least one image. In some embodiments, the n images comprise a number of images ranging between 1 and 10. In some embodiments, the n images comprise a number of images ranging between 6 and 45. In some embodiments, the method 200 comprises capturing the n images of a tongue of a subject. In some embodiments, the method 200 comprises receiving n images from an image capturing device directly and/or indirectly. In some embodiments, the method 200 comprises communicating with the image capturing device and/or with an image capturing module 106.

In some embodiments, the method 200 comprises obtaining one or more image of the tongue of a subject with a specified band of illumination. In some embodiments, the band of illumination comprises a plurality of wavelengths. In some embodiments, the band of illumination comprises wavelengths ranging between about 380-730 nm, for example about 400 nm-700 nm.

In some embodiments, at step 202, the method 200 comprises receiving n images, each depicting a tongue of a subject. In some embodiments, the method comprises obtaining the n images via a connector coupled to an imaging device. In some embodiments, the method 200 comprises receiving the n images from the image capturing module 106 and/or from the storage module 104. In some embodiments, the n images are transferred from the image capturing module 106 to one or more of the processor 102, the storage module 104, the image processing module 108, the machine learning model 110, and the user interface module 112.

In some embodiments, the method 200 comprises identifying a tongue of a subject and/or a position of a tongue of a subject in at least one of the n images. In some embodiments, the method 200 comprises identifying the tongue of a subject and/or a position of a tongue of a subject in real time. In some embodiments, the method 200 comprises identifying a position of the tongue using one or more motion sensors.

In some embodiments, the method 200 comprises capturing an image of a tongue of a subject at a predetermined position. In some embodiments, the method 200 comprises capturing an image of a tongue of a subject with a specified illumination setting. In some embodiments, the method comprises commanding the image capturing module 106 to capture an image at a specified time and/or in real time corresponding with one or more identified position of a tongue of the subject.

In some embodiments, the processor 102 is configured to identify the predetermined position of the tongue. In some embodiments, the processor 102 is configured to identify the specified illumination setting. In some embodiments, the processor 102 is configured to identify a position of the tongue in which the dorsal surface of the tongue is exposed. In some embodiments, the predetermined position comprises a position of the tongue in which any one or more of the apex of the tongue, the body of the tongue, the median lingual sulcus, the vallate papillae, and the foliate papillae of the tongue are exposed. In some embodiments, the predetermined position of the tongue comprises a position in which the tongue of the subject is extended outward. In some embodiments, the predetermined position of the tongue comprises a position in which at least a portion of the dorsal surface of the tongue is parallel to the image capturing device.

According to some embodiments, the predetermined position of the tongue is associated with visually exposing segments of the tongue in accordance with a specified tongue reflexology segment of the tongue. In some embodiments, the method 200 comprises identifying one or more tongue reflexology segments of the tongue of the subject.

In some embodiments, the method 200 comprises applying a calibration processes configured for capturing an image of the tongue. In some embodiments, one or more of the calibration processes comprises at least one of image segmentation, real time image calibration, avoiding illumination clipping, avoiding illumination clipping in real time, avoiding motion blur, and avoiding motion blur in real time. In some embodiments, each possibility is a separate embodiment.

In some embodiments, the method 200 comprises verifying the illumination uniformity of the image during and/or after the capturing of the one or more n images. In some embodiments, the method 200 comprises verifying the focus level of the image during and/or after the capturing to the one or more n images. In some embodiments, the method 200 comprises controlling the shading of a lens of the image capturing device. In some embodiments, the method 200 comprises calibrating the image captured by the image capturing device. In some embodiments, the method 200 comprises calibrating the n images.

In some embodiments, method 200 comprises illuminating the tongue of the subject with a specified illumination. In some embodiments, the method 200 comprises illuminating the tongue of the subject such that the tongue of the subject is obtained with an optimal color fidelity. In some embodiments, the method 200 comprises calibrating the specified illumination prior to obtaining the image data. In some embodiments, the method 200 comprises continuously calibrating the specified illumination. In some embodiments, the method 200 comprises adjusting an illumination type, the illumination spectrum, and/or a position of the illuminating element of the image capturing device in order to maintain a metameric match between the colors of the tongue of the subject and the captured n images.

In some embodiments, the method 200 comprises capturing a video and/or a plurality of images. In some embodiments, the method 200 comprises uploading the captured image, images, and/or video to a cloud storage unit. In some embodiments, the method 200 comprises analyzing a motion vector of the tongue depicted by the video and/or the plurality of images. In some embodiments, the method 200 comprises tracking the tongue. In some embodiments, the method 200 comprises tracking the tongue in real time and/or in a captured video segment. In some embodiments, the method 200 comprises analyzing a vibration of the tongue. In some embodiments, the method 200 comprises analyzing a motion vector of the tongue. In some embodiments, the method 200 comprises analyzing a movement associated with movements of the image capturing device. In some embodiments, the method comprises differentiating between movements associated with tongue vibrations and movements associated with movements of the image capturing device.

A potential advantage to analyzing a movement of the tongue is in that the analysis allows removal of blurring caused by vibrations of the tongue and enables the generation of a clearer image.

In some embodiments, the method 200 comprises motion detection wherein images captured during movement are assigned one or more motion vectors. In some embodiments, the method comprises sorting out of detected images in which the vector exceeds a predetermined threshold value. In some embodiments, the predetermined threshold value corresponds to a predetermined resolution of the processed image.

In some embodiments, at step 204, the method 200 comprises preprocessing the n images, thereby obtaining n′ images. In some embodiments, the preprocessing comprises at least one of image selection and image adjustment. In some embodiments, the preprocessing comprises normalization of the n images. In some embodiments, the image adjustment comprises adjustment of one or more of contrast, brightness, level, hue, and saturation of the n images. In some embodiments, each possibility is a separate embodiment. In some embodiments, the method comprises pre-processing at least a portion of the n images.

According to some embodiments, the preprocessing comprises generating segmentations representing specified areas of the tongue within the n images. According to some embodiments, one or more of the specified areas of the tongue may be associated with one or more rejoin of the gastrointestinal tract. According to some embodiments, one or more of the specified areas of the tongue may correspond to one or more pathologies of the gastrointestinal tract. According to some embodiments, the specified areas of the tongue each correspond to individual pathologies of the gastrointestinal tract.

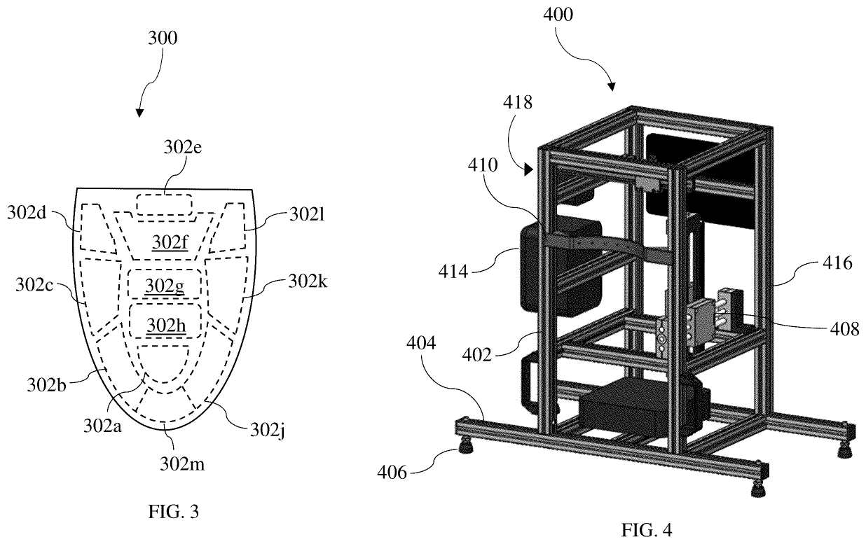

Reference is made to FIG. 3, which is a front view schematic illustration of an exemplary segmentation map, in accordance with some embodiments of the present invention. According to some embodiments, the segmentations 302a/302b/302c/302d/302e/302f/302g/302h/302i/302j/302k/302l/302m (collectively referred to hereinafter as segmentations 302) define a map 300 of at least a portion of a surface of the tongue of the subject. According to some embodiments, the segmentations 302 define at least 2, 3, 5, 8 or 10 specified areas. According to some embodiments, two or more segmentations 302 are adjacent. According to some embodiments, two or more segmentations 302 are congruent. According to some embodiments, two or more segmentations 302 coincide.

According to some embodiments, the preprocessing comprises assigning a key to each of the segments of the segmentations 302, such as, for example, a number value and/or color. According to some embodiments, the key is indicative to a certain area of the tongue, for example, e.g. tongue tip, base of the tongue. According to some embodiments, the map 300 of the segmentations 302 can be represented by the keys associated with the segmentations 302. According to some embodiments, the preprocessing comprises mapping the tongue of the subject based on, at least in part, the segmentations 302 and the associated key of each of the segmentations.

According to some embodiments, the method 200 comprises inputting the segmentations 302 and the keys associated with the segmentations 302 in the machine learning module. According to some embodiments, method 200 comprises assigning an indicative value to the inputted keys, wherein the indicative value is associated with a specific pathology. According to some embodiments, the method 200 comprises inputting the indicative value to the machine learning module. According to some embodiments, the indicative value of the key is associated with a relevance of the specific pathology to the specified area of the tongue associated with the key. According to some embodiments, the indicative value is binary. According to some embodiments, the indicative value comprises a range corresponding to a level of relevance of a specified area with a specific pathology.

For example, for a detection of a certain pathology or risk for malignancy thereof, there may be only one or two relevant segmentations 302, such as segments 302a and 302j. In such an example, the indicative value assigned to other segments, such as 302d and 302k, may be “0”, while the indicative value assigned to 302a and 302j may be “1”.

According to some embodiments, the machine learning module is configured to analyze the n images in accordance with the indicative value assigned to the keys and/or segmentations 302. According to some embodiments, the machine learning module is configured to overlook segmentations of which the indicative value associated with the key corresponds to a low relevance level of a specific pathology. For example, in some embodiments, the machine learning module is configured to overlook segmentation of which the indicative value associated with the key is “0”.

In some embodiments, the method 200 comprises assessing the quality of the n images. In some embodiments, the processor 102 receives data of the quality of the n images. In some embodiments, the method 200 comprises commanding a change in the image capturing module 106 based on the assessment. In some embodiments, for images in which a change of the ambient conditions during the capturing of the image may enhance the quality of the outcome for the images, the processor 102 can command a change in the image capturing module 106 such that a change is made to the ambient conditions. For example, the processor may command a change of the brightness surrounding the subject while capturing the images. In some embodiments, the method 200 comprises commanding a user to make a change of position and/or lighting using the user interface module 112. In some embodiments, and as described in greater detail elsewhere herein, the processor 102 is configured to directly command a change of the image capturing device during the capturing of the n images.

In some embodiments, the method 200 comprises adjusting any one or more of the angle of the image capturing device, the light emitted from the illumination element of the image capturing device, the brightness of the illumination of the subject during the image capturing, the angle of the image capturing device in relation to the tongue of the subject, and the exposure time used during the image capturing. In some embodiments, the method comprises adjusting any one or more of the settings of the image capturing module and/or camera, the background, background, magnifications, and magnification of specified areas of the tongue and/or oral cavity of the subject.

In some embodiments, the method 200 comprises combining two or more of the n images to form a single image of the n′ images. In some embodiments, the method comprises combining a plurality of the n images to form a plurality of n′ images. In some embodiments, the method comprises image stitching. In some embodiments, the method comprises stitching the n images to obtain the n′ images. In some embodiments, the method comprises stitching the n images. In some embodiments, the method comprises stitching the n images such that a single stitched image comprises data of different angles of the tongue which were each depicted by separate images of the n images. In some embodiments, the method comprises generating the n′ images from the n images such that the n′ images comprise super resolution images. In some embodiments, the number of n′ images corresponding with the n images is smaller than the number of the obtained n images. In some embodiments, the number of n′ images corresponding with the n images is equal to the number of the obtained n images.

In some embodiments, the number of n′ images corresponding with the n images is larger than the number of the obtained n images. In some embodiments, the n′ images comprise data from one or more of the n images, for example, depicting a specific feature of the n images. In some embodiments, the n′ images comprise partial data from one or more of the n images. In some embodiments, one or more of the n′ images comprise partial data from a plurality of n images.

In some embodiments, the method 200 comprises obtaining high dynamic range (HDR) imaging using the n images. In some embodiments, the method 200 comprises image adjustment comprising creating one or more images from at least a portion of the n images. According to some embodiments, the n images are obtained using one or more different focus or light exposure levels. According to some embodiments, at least a portion of the n images are combined into one or more images, thereby obtaining optimal exposure for each portion of the tongue of the subject in one or more images.

In some embodiments, the number of n′ images corresponding with the n images is smaller than the number of obtained n images due to sorting of the n images. In some embodiments, one or more of the n images is not associated with the n′ images due to any one or more of a resolution lower than a predetermined threshold, a contrast lower than a predetermined threshold, and a contrast higher than a predetermined threshold. In some embodiments, the processor 102 determined the predetermined threshold. In some embodiments, a user can determine the predetermined threshold via the user interface module 112.

In some embodiments, the method 200 comprises applying image processing algorithms to the n′ images. In some embodiments, at step 206, the method 200 comprises producing m presentations of each of the n′ images. In some embodiments, the method 200 comprises producing m presentations based on the n′ images. In some embodiments, the method 200 comprises producing m presentations of each of the n′ images using at least one feature enhancing and/or extracting algorithm.

In some embodiments, one or more of the m presentations comprises one or more features extracted from the n′ images. In some embodiments, one or more of the m presentations comprises one or more of the n images. In some embodiments, the m presentations may be images. In some embodiments, the m presentations may include files in one or more image formats.

In some embodiments, one or more of the m presentations comprises a tongue segmentation color map, motion vectors associated with the n images, two dimensional representations of the tongue of the subject, three dimensional representations of the tongue of the subject, representations of different planes of the tongue, a plurality of positions of the tongue, a topological map of the tongue, and one or more digitally generated video segments. In some embodiments, the m presentations comprise positions and/or configurations of the tongue of a subject digitally generated without an image of the tongue in the particular position represented in the presentations. In some embodiments, the m presentations comprise a function of time.

In some embodiments, one or more of the m presentations comprises one or more features extracted from the n′ images. In some embodiments, the extracted features include colors, morphology of one or more of the surfaces of the tongue, topology of the tongue, dimensions of the tongue, and vibration analysis of the tongue. In some embodiments, each possibility is a separate embodiment.

In some embodiments, at step 208, the method 200 comprises classifying the n′*m presentations into classes, wherein the classes comprise at least a positive for lower gastro-enteral pathology and a negative for lower gastro-enteral pathology. According to some embodiments, classifying of the n′*m presentations includes applying a trained machine learning module 110 on the n′*m presentations. In some embodiments, the classes comprise at least a positive for gastrointestinal disorders and a negative for gastrointestinal disorders. In some embodiments, the method comprises classifying the subjected based on each of the n′ images individually. In some embodiments, the method comprises classifying the subject based on each of the m presentations individually.

In some embodiments, the method 200 comprises further subclassifying the subject who is classified as being positive for gastrointestinal disorders into one or more subclassifications of colon-related pathology and gastro-related pathology. In some embodiments, the method 200 comprises subclassifying a colon-related pathology into one or more of colorectal carcinoma (CRC), polyps, inflammatory bowel disease (IBD) involving the lower intestinal tract (colon). In some embodiments, the method 200 comprises subclassifying an upper gastrointestinal pathology into one or more of gastric malignancy, gastritis, esophageal malignancy, esophagitis and duodenitis.

In some embodiments, at step 210, the method 200 comprises identifying the subject as suffering from a gastrointestinal disorder. In some embodiments, the method comprises identifying the subject as suffering from a gastrointestinal disorder based, at least in part, on the percent of the n′*m presentations associated with a subject being positive for one or more gastrointestinal disorders. In some embodiments, the method comprises identifying the subject as suffering from a subclassification of gastrointestinal disorder based, at least in part, on the percent of the n′*m presentations associated with a subject being positive for the specific subclassification. In some embodiments, the method comprises identifying the subject as suffering from a gastrointestinal disorder based, at least in part, on the fraction of the n′*m presentations that are associated with each of the subclassifications.

In some embodiments, the method 200 comprises presenting the detected pathology to a user using the user interface module 112.

### Machine Learning Module and Training

In some embodiments, the machine learning module 110 comprises a machine learning module that has been trained to detect one or more gastrointestinal disorders based on one or more images of a tongue of subjects identified as suffering of one or more gastrointestinal disorders. In some embodiments, the machine learning model is trained to detect one or more gastrointestinal disorders based, at least in part, on meta data associated with the subject, such as, for example, one or more of age, gender, blood pressure, eating habits, and medical history of the subject. In some embodiments, the sensitivity of the detection is above at least one of 70%, 80% 90%, 95%, and 98%. In some embodiments, each possibility is a separate embodiment.

In some embodiments, the machine learning model uses all of the n images and/or the m presentations to detect the gastrointestinal disorders. In some embodiments, the machine learning module uses all of the meta data to detect the gastrointestinal disorders. In some embodiments, the machine learning module comprises at least one neural network configured to use all of the received data in order to detect one or more gastrointestinal disorder and/or identify the pathology associated with the subject.

In some embodiments, the machine learning model is trained on a training set comprising m presentations associated with n images of subjects having identified gastrointestinal disorders. In some embodiments, the machine learning model is trained on a training set comprising m presentations associated with n images of subjects identified as not suffering from gastrointestinal disorders. In some embodiments, the machine learning model is trained on a training set comprising m presentations associated with n′ images of subjects having identified gastrointestinal disorders. In some embodiments, the machine learning model is trained on a training set comprising m presentations associated with n′ images of subjects identified as not suffering from gastrointestinal disorders. In some embodiments, the machine learning model is trained on a training set comprising the n′*m presentations of subjects which have identified gastrointestinal disorders. In some embodiments, the m presentations of the training set comprise labels indicating at least a positive for gastrointestinal disorders and a negative for gastrointestinal disorders. In some embodiments, the labels indicate any one or more of a colon-related pathology, lower gastrointestinal related pathology, upper gastrointestinal related pathology, upper gastrointestinal related pathology colorectal carcinoma (CRC), polyps, inflammatory bowel disease (IBD), gastric malignancy, gastritis, esophageal malignancy, esophagitis and duodenitis.

In some embodiments, a same machine learning model is trained for a plurality of gastrointestinal disorders. In some embodiments, the same machine learning model is trained using labels associated with a plurality of gastrointestinal disorders.

In some embodiments, the labels are associated with different segments of the tongue depicted by the n′*m presentations. In some embodiments, a single image is associated with a plurality of labels wherein each label is associated with a different segment of the tongue depicted by the image. In some embodiments, the labels are associated with segments of the tongue depicted by the n′*m presentations. In some embodiments, a single image is associated with a plurality of labels wherein each label is associated with a tongue reflexology segment of the tongue of the subject depicted.

### Imaging Capturing Device

Reference is made to FIG. 4, which shows a perspective view simplified illustration of an exemplary image capturing device for detection of gastrointestinal disorders, in accordance with some embodiments of the present invention. In some embodiments, the image capturing device 400 is configured to enable a uniformity in the positioning of the tongue of the subject in relation to the camera and/or lends of the image capturing device 400. In some embodiments, the image capturing device 400 is in operable communication with at least one of the image capturing module 106 and the processor 102. In some embodiments, each possibility is a separate embodiment. In some embodiments, the image capturing device 400 is configured to enable a user to capture an image of the tongue of a subject.

In some embodiments, the image capturing device 400 comprises a frame 402. In some embodiments, the frame 402 comprises a base 404 and at least one leg 406. In some embodiments, the frame 402 is rigid. In some embodiments, the base 406 and/or legs 406 are configured to stabilize the frame 402 when positioned on a surface. In some embodiments, the frame 402 comprises two or more posts 416 extending from one or more of the base 404 and the legs 406. In some embodiments, the two or more posts 416 are configured to support at least one of a rest 410 and a holder 408. In some embodiments, each possibility is a separate embodiment.

In some embodiments, the rest 410 comprises a forehead rest and is configured to maintain a position of a head of a subject stationary in relation to the frame 402. In some embodiments, the rest 410 is configured to abut a portion of a face of a subject. In some embodiments, the rest 410 is coupled to one post 416 at one end thereof and to a second post 406 at a second end thereof. In some embodiments, the rest 410 is rigid, semi-rigid, or flexible. In some embodiments, the rest 410 is malleable such that specific facial features of a subject can be accommodated during use. In some embodiments, the position of the rest 410 is adjustable in relation to the frame 402 and/or at least one post 416.

In some embodiments, the image capturing device 400 comprises a holder 408 configured to fix a position of a camera and/or sensor. In some embodiments, the image capturing device 400 comprises a camera and/or sensor. In some embodiments, the holder 408 comprises a dock onto which a camera and/or sensor is coupled. In some embodiments, the dock is configured to receive a generic camera, such as, for example, a smartphone. In some embodiments, the dock is slidably coupled to the frame 402. In some embodiments, the holder 408 and/or the dock is slidable about at least two axes of movement. In some embodiments, the holder 408 and/or the dock is slidable about at least three axes of movement. In some embodiments, the dock is tiltable such that the spatial orientation of the camera and/or sensor coupled to the holder 408 is changed. In some embodiments, the dock is adjustable such that the camera and/or sensor coupled to the holder 408 is tilted, panned, and/or rolled. In some embodiments, the camera is configured to pan, tilt and/or roll in relation to any one or more of the frame 402, the rest 410 and the holder 408. In some embodiments, the angle of the dock in relation to the frame 402 and/or the rest 410 is adjustable. In some embodiments, the holder 408 comprises a motor configured to drive the holder 408 into a predetermined position and/or angle in relation to the frame 402.

In some embodiments, the image capturing device 400 comprises a processor 418. In some embodiments, the image capturing device 400 is in operative communication with processor 102/418. In some embodiments, the image capturing device 400 comprises a power unit 414 in communication with the processor 102/418. In some embodiments, the power unit 414 is coupled to the motor of the holder 408.

In some embodiments, the processor 102/400 is configured to directly command a change of the structure of the image capturing device 400 during the capturing of the n images. In some embodiments, the processor 102/400 is configured to directly command a change of the position and/or angle of the holder 408 in relation to the frame 402.

Unless otherwise defined the various embodiment of the present invention may be provided to an end user in a plurality of formats, platforms, and may be outputted to at least one of a computer readable memory, a computer display device, a printout, a computer on a network, a tablet or a smartphone application or a user. Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “processor” or “computer” on a “computer network”, it should be noted that optionally any device featuring a data processor and/or the ability to execute one or more instructions may be described as a computer, including but not limited to a PC (personal computer), a server, a minicomputer, a cellular telephone, a smart phone, a PDA (personal data assistant), a pager. Any two or more of such devices in communication with each other, and/or any computer in communication with any other computer, may optionally comprise a “computer network”.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

The invention may be described in the general context of computer-executable instructions, such as program modules, being executed by a computer. Generally, program modules include routines, programs, objects, components, data structures, and so forth, which perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

While a number of exemplary aspects and embodiments have been discussed above, those of skill in the art will recognize certain modifications, additions and sub combinations thereof. It is therefore intended that the following appended claims and claims hereafter introduced be interpreted to include all such modifications, additions and sub-combinations as are within their true spirit and scope.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1.-17. (canceled)

18. A system rising:
at least one hardware processor; and
a non-transitory computer-readable storage medium having stored thereon program code, the program code executable by the at least one hardware processor to:
receive n images, each depicting at least a portion of a tongue of a subject;
preprocess the n images, wherein the preprocessing comprises at least one of image selection and image adjustment, thereby obtaining n′ images;
produce m presentations of each of said n′ images using at least one feature enhancing algorithm;
classify the produced n′*m presentations into classes, by applying a machine learning algorithm on the n′*m presentations, wherein the classes comprise at least a positive for gastrointestinal disorders and a negative for gastrointestinal disorders; and
identify the subject as suffering from a gastrointestinal disorder when at least a predetermined fraction/percentage of the produced n′*m presentations are classified as being positive for gastrointestinal disorders.

19. The system according to claim 18, wherein the image selection comprises movement detection wherein images captured during movement are assigned one or more motion vectors, followed by a sorting out of detected images in which said vector exceeds a predetermined threshold value.

20. The system according to claim 18, wherein the image adjustment comprises adjustment of one or more of contrast, brightness, level, hue, sharpness, and saturation of the n′ images.

21. The system according to claim 18, wherein the program code is executable to further subclassify the subject based, at least in part, on the n′*m presentations which are classified as being positive for gastrointestinal disorders into one or more subclassifications of colon-related pathology and gastro-related pathology.

22. The system according to claim 21, wherein the subclassification further comprises two or more subclasses of colon-specific pathologies.

23. The system according to claim 22, wherein two or more subclasses of colon-specific pathologies are selected from colorectal carcinoma (CRC), polyps, different types of polyps, and inflammatory bowel disease involving the lower intestinal tract (IBD).

24. The system according to claim 23, wherein the subclasses of the colon-specific pathologies are selected from adenomatous polyp, hyperplastic polyp, serrated polyp, inflammatory polyp, and villous adenoma polyp, and complex polyp.

25. The system according to claim 22, wherein the subclassification further comprises two or more subclasses of upper gastrointestinal specific pathologies.

26. The system according to claim 25, wherein two or more subclasses of upper gastrointestinal-specific pathologies are selected from gastric malignancy, gastritis, esophageal malignancy, esophagitis and duodenitis.

27. The system according to claim 22, wherein the subclassification comprises a score associated with a level of malignancy of a disorder.

28. The system according to claim 22, wherein the subclassification comprises a score corresponding with a potential chance of the subject developing malignancy in one or more pathologies.

29. The system according to claim 18, wherein said m presentations can additionally comprise three dimensional presentations of the depicted tongue of the subject.

30. The system according to claim 18, wherein said program is configured to receive said n images from a plurality of different types of image capturing devices.

31. The system according to claim 30, wherein said program is executable to normalize said received images.

32. The system according to claim 18, wherein said hardware processor is couplable to at least one image capturing device and said program code is executable to identify a tongue of a subject in real time.

33. The system according to claim 32, wherein said program code is executable to capture said n images.

34. A computer program product comprising a non-transitory computer-readable storage medium having program code embodied therewith, the program code executable by at least one hardware processor to:
receive n images, each depicting at least a portion of a tongue of a subject;
preprocess the n images, wherein the preprocessing comprises at least one of image selection and image adjustment, thereby obtaining n′ images;
produce m presentations of each of said n′ images using at least one feature enhancing algorithm;
classify the produced n′*m presentations into at least two classes by applying a trained machine learning algorithm on the n′*m presentations, wherein the at least two classes comprise positive for gastrointestinal disorders and negative for gastrointestinal disorders; and
identify the subject as suffering from a gastrointestinal disorder when at least a predetermined fraction/percentage of the produced n′*m presentations are classified as being positive for gastrointestinal disorders.
